(19) United States
(12) Patent Application Publication
(10) Pub. No.: US 2005/0187170 A1
(43) Pub. Date: Aug. 25, 2005

(54) Enhancing the efficiency of RNA polymerase inhibitors by using inosine monophosphate dehydrogenase inhibitors

(75) Inventors: Shanta Bantia, Birmingham, AL (US); Yarlagadda S. Babu, Birmingham, AL (US)

Correspondence Address: CONNOLLY BOVE LODGE & HUTZ LLP, SUITE 800, 1990 M STREET NW, WASHINGTON, DC, 20036-3425, US

(73) Assignee: BIOCRYST PHARMACEUTICALS, INC., Birmingham, AL (US)

(21) Appl. No.: 10/867,683

(22) Filed: Jun. 16, 2004

Related U.S. Application Data
(60) Provisional application No. 60/478,357, filed on Jun. 16, 2003.

(51) Int. Cl. A61K 31/7056; A61K 31/704; A61K 31/496; A61K 31/409

(52) U.S. Cl. 514/34; 514/43; 514/44; 514/410; 514/252.13; 514/449

(57) ABSTRACT

The present invention relates generally to pharmaceutical compositions useful to treat viral diseases. The present invention relates particularly RNA polymerase inhibitors and inosine monophosphate dehydrogenase (IMPDH) inhibitors such as mycophenolate compounds.

### REFERENCE TO RELATED APPLICATION

This Application claims the priority of provisional application 60/478,357, filed Jun. 16, 2003, the entire contents of the recited priority document is hereby specifically incorporated by reference, in its entirety, for all purposes.

### FIELD OF THE INVENTION

The present invention relates generally to treating viral diseases. The present invention relates particularly to enhancing the efficiency of RNA polymerase inhibitors by using inosine monophosphate dehydrogenase (IMPDH) inhibitors such as mycophenolate compounds.

### BACKGROUND

The information provided below is not admitted to be prior art to the present invention, but is provided solely to assist the understanding of the reader.

Hepatitis C is a global problem of immense importance. Currently, worldwide, there are an estimated 170 million people infected with hepatitis C virus (HCV). The consequences of chronic hepatitis C include chronic disease, cirrhosis, and hepatocellular carcinoma. However, the course of this infection is variable and host, viral, or environmental factors may be associated with progression of the disease. For example, women appear to have a relatively slowly progressive course, and alcohol consumption seems to facilitate the disease progression.

Strides continue to be made in the treatment of chronic hepatitis C. The current standard of care is combination therapy with interferon alfa/Pegylated interferon and ribovirin, which achieves an approximately 50%-80% sustained response rate. Response rates are dependent on several host and viral factors: women, particularly those younger than 40 years, have a better rate; HCV genotypes 2 and 3 respond better than genotype 1; and low viral load and low body weight also correlate with a favorable response to therapy.

The current treatment regimen of interferon alfa/PEG interferon plus ribavirin is associated with a variety of side effects that has led to decreases in drug doses and even discontinuation. Combination interferon and ribavirin therapy is associated with adverse experiences that may include “flu-like symptoms” psychiatric episodes of depression, psychosis, aggressive behavior, hallucinations, and suicidal attempts. More specifically related to ribavirin use, anemia can be found to lead to exacerbation of coronary artery disease symptoms. The precise mechanism of action of both interferon and ribavirin is unknown. Considerable effort is being expended to develop newer therapies with an understood mechanism of action to treat hepatitis C.

The synthesis of nucleotides in organisms is required for the cells in those organisms to divide and replicate. Nucleotide synthesis in mammals may be achieved through one of two pathways: the de novo synthesis pathway or the salvage pathway. Different cell types use these pathways to a different extent.

Inosine-5′-monophosphate dehydrogenase (IMPDH; EC 1.1.1.205) is an enzyme involved in the de novo synthesis of guanosine nucleotides. IMPDH catalyzes the NAD-dependent oxidation of inosine-5′-monophosphate (IMP) to xanthosine-5′-monophosphate (XMP)[Jackson R. C. et. al., Nature, 256, pp. 331-333, (1975)].

Two isoforms of human IMPDH, designated type I and type II, have been identified and sequenced [F. R. Collart and E. Huberman, J. Biol. Chem., 263, pp. 15769-15772, (1988); Y. Natsumeda et. al., J. Biol. Chem., 265, pp. 5292-5295, (1990)]. Each is 514 amino acids, and they share 84% sequence identity. Both IMPDH type I and type II form active tetramers in solution, with subunit molecular weights of 56 kDa [Y. Yamada et. al., Biochemistry, 27, pp. 2737-2745 (1988)].

The de novo synthesis of guanosine nucleotides, and thus the activity of IMPDH, is particularly important in B and T-lymphocytes. Thus, IMPDH is an attractive target for selectively inhibiting the immune system without also inhibiting the proliferation of other cells.

It is also known that IMPDH plays a role in other metabolic events. Increased IMPDH activity has been observed in rapidly proliferating human leukemic cell lines and other tumor cell lines, indicating IMPDH as a target for anti-cancer as well as immunosuppressive chemotherapy [M. Nagai et. al., Cancer Res., 51, pp. 3886-3890, (1991)]. IMPDH has also been shown to play a role in the proliferation of smooth muscle cells, indicating that inhibitors of IMPDH, such as MPA or rapamycin, may be useful in preventing restenosis or other hyperproliferative vascular diseases [C. R. Gregory et al., Transplantation, 59, pp. 655-61 (1995); PCT publication WO 94/12184; and PCT publication WO 94/01105].

Additionally, IMPDH has been shown to play a role in viral replication in some viral cell lines. [S. F. Carr, J. Biol. Chem., 268, pp. 27286-27290 (1993)]. Analogous to lymphocyte and tumor cell lines, the implication is that the de novo, rather than the salvage, pathway is critical in the process of viral replication.

Mycophenolic acid (MPA) is a potent inhibitor of IMPDH (Carr, et al. J. Biol. Chem. 1993, 268, 27286-27290). It blocks B and T lymphocyte proliferation and has been used as an immunosuppressant (Wu, J. C. In Perspectives in Drug Discovery and Design, Wyvratt, M. J.; Sigal, N. H., Eds.; ESCOM Science Publ., Leiden, 1994, Vol. 2, pp 185-204), although it is inactive against tumors due to its quick conversion into the inactive.beta.-glucuronide after administration (Franklin, et al. Cancer Res., 1996, 56, 984-987). MPA inhibits IMPDH with even better specificity against the type II isoform dominant in cancer cells (Kᵢ=6-10 nM) than type I expressed in normal cells (Kᵢ=33-37 nM) (Carr, et al.). When the MPA binds to the cofactor moiety of IMPDH, it resembles that of nicotinamide mononucleotide (NMN) with a carboxyl group positioned at the space occupied by the phosphoryl group of NMN (Sintchak, et al., Cell, 1996, 85, 921-930).

U.S. Pat. Nos. 5,380,879 and 5,444,072 and PCT publications WO 94/01105 and WO 94/12184 describe mycophenolic acid (MPA) and some of its derivatives as potent, uncompetitive, reversible inhibitors of human IMPDH type I (Kᵢ=33 nM) and type II (Kᵢ=9 nM).

Nucleoside analogs such as tiazofurin, ribavirin and mizoribine also inhibit IMPDH [L. Hedstrom, et. al. Biochemistry, 29, pp. 849-854 (1990)]. These compounds, which are competitive inhibitors of IMPDH, suffer from lack of specificity to this enzyme.

The IMPDH inhibitor ribavirin is currently used for the treatment of hepatitis C virus (HCV) infection and disease. Ribavirin enhances the sustained efficacy of interferon in HCV treatment. However, the therapeutic potential of ribavirin is limited by its lack of a sustained response in monotherapy and broad cellular toxicity.

Nucleosides derived from natural D-ribose play a significant role for the treatment of human viral diseases, neoplastic diseases, and modulation of immune response. Among them, Ribavirin (1-beta-D-ribofuranosyl-1,2,4-triazole-3-carboxamide), AZT (3′-azido-3′-deoxythymidine), ddI (2′,3′-dideoxyinosine), and ddC (2′,3′-dideoxycytidine) are among the most prominent drugs presently approved. Despite their relatively potent antiviral and antineoplastic activity, emerging resistance of many viruses and tumor cells prompted a search for new nucleosides.

Ribavirin, a purine analog, is the drug of choice in treating respiratory syncytial viruses (RSV) infection. This compound appears to act by reducing cellular GTP levels, blocking the action of several GTP-dependent viral processes.

Inosine monophosphate dehydrogenase inhibitors like mycophenolic acid and ribavirin are known to alter both nucleotide and deoxynucleotide pools. Mycophenolic acid (MPA), an inhibitor of inosine monophosphate dehydrogenase, shows strong anti-HIV activity in vitro in both human peripheral blood CD4+ lymphocytes and macrophages, as well as established human cell lines. MPA shows its greatest antiviral effects during the early stages of HIV infection. By limiting the rate of de novo synthesis of guanosine nucleotides, the drug apparently blocks the activity of reverse transcriptase, which is required for the formation of the HIV DNA provirus. MPA (at concentrations that do not affect proliferation) when combined with ABC or DDI, synergistically enhances activity against wild-type HIV and the NRTI-resistant HIV clone DRSM34. MPA also enhances the activity of TFV against both wild-type HXB2 and TFV-resistant strain HIV (K65R), in a more than additive manner. Since MPA and ribavirin alters the nucleotide and deoxynucleotide pools and mainly decreases the formation of guanosine and deoxyguanosine nucleotides it should potentially enhance the activity of RNA polymerase inhibitors mainly nucleosides and non-nucleosides (as they bind to the nucleotide binding site). MPA in combination with RNA polymerase inhibitors mainly nucleosides and non-nucleosides (as they bind to the nucleotide binding site) provides a novel strategy.

### SUMMARY OF INVENTION

An aspect of the present invention provides a pharmaceutical composition comprising: an effective amount of at least one RNA polymerase (RNAP) inhibitor; and an effective amount of at least one inosine monophosphate dehydrogenase (IMPDH) inhibitor, wherein the amount of the IMPDH inhibitor may be less than, equal to, or more than the amount RNAP inhibitor and the combination provides an effective treatment for viral infections.

An aspect of the present invention provides the IMPDH inhibitor is a mycophenolate or ribavirin.

An aspect of the present invention provides a method of treating a viral infection comprising co-administration of at least one RNA polymerase (RNAP) inhibitor; and an effective amount of at least one inosine monophosphate dehydrogenase (IMPDH) inhibitor.

An aspect of the present invention provides a method of treating a viral infection comprising administration of at least one RNA polymerase (RNAP) inhibitor prior to administration of an effective amount of at least one inosine monophosphate dehydrogenase (IMPDH) inhibitor.

An aspect of the present invention provides a method of treating a viral infection comprising administration of at least one inosine monophosphate dehydrogenase (IMPDH) inhibitor prior to administration of an effective amount of at least one RNA polymerase (RNAP) inhibitor.

An aspect of the present invention provides a method of treating a viral infection comprising: administering to infected cells a pharmaceutical composition comprising an effective amount of at least one RNAP inhibitor and at least one mycophenolate.

An aspect of the present invention provides a method of treating a viral infection comprising: administering to infected cells a pharmaceutical composition comprising an effective amount of at least one RNAP inhibitor and ribavirin.

An aspect of the present invention provides a method of manufacturing a pharmaceutical composition comprising bringing into association with a pharmaceutical carrier a) a first amount of at least one RNA polymerase inhibitor; and b) a second amount of at least one inosine monophosphate dehydrogenase inhibitor, wherein the inosine monophosphate dehydrogenase inhibitor is a mycophenolate or ribavirin, and wherein said second amount is less than, equal to, or more than said first amount and the combination provides an effective treatment for a viral infection.

### DETAILED DESCRIPTION OF A PREFERRED EMBODIMENT

As used herein, the term “viral infection” refers to any stage of a viral infection, including incubation phase, latent or dormant phase, acute phase, and development and maintenance of immunity towards a virus. Consequently, the term “treatment’ is meant to include aspects of generating or restoring immunity of the patient's immune system, as well as aspects of suppressing or inhibiting viral replication.

The term “mycophenolate” refers to mycophenolic acid and analogs and their pharmaceutically acceptable salts, derivatives, and prodrugs.

With regard to the mycophenolate compounds for use in the pharmaceutical combinations of the present invention, “pharmaceutically acceptable derivatives” may preferably be esters, and more preferably heterocycloalkyl esters, for example, morpholinoethyl esters.

As used herein, “MA” refers specifically to the 2-morpholinoethyl ester of mycophenolic acid, mycophenolate mofetil (Cellcept®, Roche Laboratories).

Mycophenolic acid is a potent and lymphocyte-selective inhibitor of de novo purine synthesis. IMPDH inhibitors useful in the practice of the invention may be selected from mycophenolic acid and pharmaceutically acceptable salts, prodrugs, and derivatives, and mycophenolic acid analogs and pharmaceutically acceptable salts, prodrugs, and derivatives. Such compounds, their preparation, and their pharmaceutical compositions and dosage formulations are described in the following United States patents, the disclosures of which are hereby incorporated herein in their respective entireties:

| | | | | |
|---|---|---|---|---|
| 5,688,529 | 5,633,279 | 5,633,279 | 5,554,612 | 5,538,969 |
| 5,536,747 | 5,493,030 | 5,444,072 | 5,441,953 | 5,380,879 |
| 5,536,747 | 5,493,030 | 5,455,045 | 5,444,072 | 5,441,953 |
| 5,554,612 | 5,554,384 | 5,545,637 | 5,543,408 | 5,538,969 |
| 5,380,879 | 5,247,083 | 4,861,776 | 4,753,935 | 4,748,173 |
| 4,727,069 | 4,725,622 | 4,686,234 | | |

Mycophenolic acid has the formula (I):

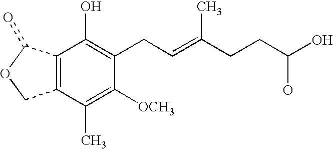

Mycophenolic acid is commercially available, e.g., from Calbiochem Corporation, Fluke Chemie AG, Indofine Chemical Company, Inc., Sigma Chemical Company, and Roche Labs.

Related compounds include mycophenolic alcohol (II):

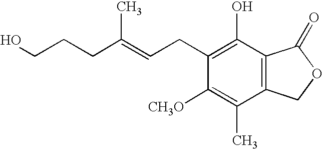

A preferred related compound is mycophenolic acid 2-morpholinoethyl ester (III):

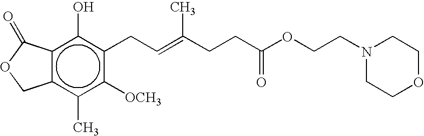

Analogs of mycophenolic acid that have high IMPDH-inhibiting activity are also useful in the practice of the present invention include compounds with varying substituents in the 4-, 5-, and 6-positions on the mycophenolate core structure, as well as pharmaceutically acceptable salts, derivatives, prodrugs, and metabolites of such mycophenolate analogs. Such compounds are described extensively in the U.S. patents tabulated above and incorporated herein by reference.

Nelson, P. H. et al. [39 J Med Chem 4181 (1996)] reported the structure-activity relationships in the region of the phthalide ring of mycophenolic acid. Replacement of the lactone ring with other cyclic moieties resulted in loss of potency, especially for larger groups. Replacement of the ring by acyclic substituents also indicated a strong sensitivity to steric bulk. A phenolic hydroxyl group, with an adjacent hydrogen bond acceptor, was found to be essential for high potency. The aromatic methyl group was essential for activity; the methoxyl group could be replaced by ethyl to give a compound with 2-4 times the potency of mycophenolic acid in vitro and in vivo.

The effects of modifying the side chain were also reviewed by the same authors [Nelson, P. H. et al., 33 J Med Chem 833 (1990)]. The side-chain appeared more intolerant of variation: twelve side-chain variants of mycophenolic acid were made either from mycophenolic acid itself or from 5-(chloromethyl)-1,3-dihydro4-hydroxy-6-methoxy-7-methyl-3-oxoisobenzofuran. Replacement of the methylated E double bond of the natural product with a triple bond, a Z double bond, a saturated bond, or a sulfur atom, with overall chain lengths equal to or greater than that of mycophenolic acid, produced compounds devoid of significant activity. Replacement of the side-chain double bond with difluoro, dibromo, or unsubstituted cyclopropane rings also removed most activity. Replacement of the double bond with an allenic linkage yielded a compound with about one-fifth of the immunosuppressive activity of mycophenolic acid.

The potential utility of the analogs is evaluated straightforwardly by skilled practitioners using cell culture assays, IMPDH-inhibition assay as described, e.g., in U.S. Pat. No. 5,633,279, and other assays known to the art.

In an especially preferred embodiment, the mycophenolate IMPDH inhibitor is mycophenolate mofetil (III), the 2-morpholinoethyl ester of mycophenolic acid (I):

Mycophenolate mofetil (Cellcept®), abbreviated “MA” herein, is the 2-morpholinoethylester prodrug of mycophenolic acid. Mycophenolate mofetil is currently approved for use in the prophylaxis of renal allograft rejection, and is in wide clinical use in solid organ transplantation. In such applications, a typical dosage is 2-3 g per day. The art recognizes classes of IMPDH inhibitors other than mycophenolates.

Compounds in classes other than mycophenolates are also suitable as the IMPDH inhibitor of the present invention. It is anticipated that IMPDH inhibitory compounds may be present as analogues, salts, and/or prodrugs. Nucleoside analogues tiazofurin, ribavirin, and mizoribine are known to the art [Hedstrom, et al., 29 Biochemistry 849 (1990); Glue, P., “The clinical pharmacology of ribavirin,” 19 SEMINARS IN LIVER DISEASE, SUPPL. 1, 17 (1999)]. VX-497 is disclosed in WO 97/41211 and WO 01/00622 (assigned to Vertex) and is further discussed in WRIGHT T. ET AL., “Dose-ranging study of VX-497, a novel oral IMPDH inhibitor, in patients with hepatitis C,” 30 HEPATOLOGY 408A (1999); ‘Broad-spectrum antiviral activity of the IMP dehydrogenase inhibitor VX-497: a comparison with ribavirin and demonstration of antiviral additivity with alpha interferon’ ANTIMICROBIAL AGENTS AND CHEMOTHERAPY vol. 44, no. 4, April 2000, pages 859-866. Brenidin is known to the art. N-[4-(5-oxazolyl)phenyl]-N,-(5-methyl-2-thiazolyl)-urea is disclosed in WO 97/40028.

The patent literature further discloses additional IMPDH inhibitors including:
ureas, U.S. Pat. No. 6,498,178;
rapamycin;
polycyclic, secondary amines, U.S. Pat. No. 6,518,291;
ureas, thioureas, and sulfonyl ureas, U.S. Pat. Nos. 6,541,496, 6,054,472, 5,807,876, 5,932,600, PCT/US97/06623;
phenyl thioureas, U.S. Pat. No. 6,555,561;
heterocyclic carboxamide-containing thioureas, U.S. Pat. Nos. 6,426,335, 6,410,571, 6,407,249;
diaminopyridine-containing thioureas, U.S. Pat. Nos. 6,403,617, 6,407,123, 6,271,236;
aminopyridine-containing thioureas, U.S. Pat. Nos. 6,380,243, 6,262,090;
acetamide-containing thioureas, U.S. Pat. No. 6,335,350;
compounds having an amine nucleus, U.S. Pat. No. 6,399,773, PCT/US99/24825;
Heterocycles, WO 01/081340, WO 00/26197, PCT/USO01/12900;
Prodrugs of carbamates (ureas), U.S. Pat. No. 6,395,763, PCT/US00/17400, WO 00/056331;
Oxamides, WO 01/107065;
tetraphosphonate bicyclic trisanhydrides, U.S. Pat. No. 6,326,490; PCT/US97/18323;
Purine analogues, PCT/US01/44709;
nucleoside analogs such as ribavarin, EICAR, Pyrazofurin, 3-deazaguanine, GR92938X and LY253963, PCT/US01/32459;
5-substituted oxazoles, PCT/US00/13817, oxazoles, PCT/US99/24889; 2-aminoquinolones, PCT/GB02/04754;
phenylenediamine containing, α-methylbenzyl thioureas, U.S. Pat. No. 6,255,349;
phenylenediamine-containing, heterocyclic carboxamide thioureas, U.S. Pat. Nos. 6,201,013, 6,197,803;
Analogues of adenosine 5′diphosphate, U.S. Pat. No. 5,700,786; and
C-nucleoside isosteres of nicotinamide adenine dinucleotide, U.S. Pat. Nos. 5,658,890, 5,569,650.

The term “analog” is intended to mean compounds derived from a particular parent compound by straightforward substitutions that do not result in a substantial (i.e. more than a factor of 100) loss in the biological activity of the parent compound, where such substitutions are modifications well-known to those skilled in the art, e.g., esterification, replacement of hydrogen by halogen, replacement of alkoxy by alkyl, replacement of alkyl by alkoxy, etc.

A “prodrug” is intended to mean a compound that is converted under physiological conditions or by solvolysis or metabolically to a specified compound that is pharmaceutically active. In the context of the mycophenolate compounds for use in the synergistic combinations of the present invention, ester derivatives are prodrugs that are metabolized to mycophenolic acid.

A “pharmaceutically active metabolite” is intended to mean a pharmacologically active product produced through metabolism in the body of a specified compound.

Any reference herein to any of the above compounds also includes a reference to a pharmaceutically acceptable salt thereof.

A “pharmaceutically acceptable salt” is intended to mean a salt that retains the biological effectiveness of the free acids and bases of the specified compound and that is not biologically or otherwise undesirable. Examples of pharmaceutically acceptable salts include but are not limited to sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caprotes, heptanoates, propioltes, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, sulfamates, xylenesulfonates, phenylacetates, phenylpropionates, phenylbutyrates, citrates, lactates, gamma-hydroxybutyrates, glycollates, tartrates, methanesulfonates, propanesulfonates, naphthalene-1-sulfonates, naphthalene-2-sulfonates, and mandelates.

Some of the compounds described herein can exist in more than one tautomeric form, all of which are intended to be comprehended.

The term “pharmaceutically acceptable derivative” is used herein to denote any pharmaceutically or pharmacologically acceptable salt, ester, amide or salt of such ester or amide of a synergistic compound according to the invention.

The term “therapeutically effective combination” is intended to mean an amount of the inventive synergistic combination that, when administered to a patient in need of treatment, is sufficient to effect treatment for the disease condition alleviated by the RNAP-IMPDH inhibitor combination. A non-limiting example of a disease condition alleviated by the RNAP-IMPDH combination is a viral infection. Amounts of each of the synergistic components present in a therapeutically effective combination may not be therapeutically effective when administered singly. The amount of a given combination that will be therapeutically effective will vary depending on factors such as the particular combination employed, the particular virus and viral strain infecting the patient, the anti-retroviral treatment history of the patient, the age and health of the patient, and other factors. Amounts of an RNAP inhibitor and an IMPDH inhibitor to comprise a therapeutically effective combination are easily determinable by medical doctors or other persons of skill in the pharmacological arts.

According to the invention, an RNA polymerase inhibitor is a compound that causes a reduction in RNA synthesis. RNAP inhibitors are generally evaluated as the ability of a test compound to inhibit the activity of NS5B polymerase assayed using two different RNA templates, including poly(A) (primer dependent) or HCV RNA templates (primer independent, includes the genomic 3′-X stem loop). The RNA products recovered represent template sized RNAs and not 3′ terminal transferase product. An HCV replicon assay is used to determine the ability of compounds to inhibit HCV RNA replication in mammalian cells. This assay has the advantage of determining if compounds traverse mammalian cell membranes and phosphorylating compounds (e.g., nucleoside analogues).

Exemplary, though non-limiting, examples of viral RNA polymerase inhibitors useful for the purposes of the present invention include: rifampin (rifampicin), amanitin, actinomycin D, streptolydigin, doxorubicin, and Tagetin™ (trademark of Epicentre, Madison Wis.).

Corallopyronin A/B and myxopyronin A/B are natural products isolated from gliding bacteria (Corallococcus coralloides; Myxococcus fulvus) and discovered to be RNAP inhibitors. Reichenbach, H., et al., Liebigs Ann. Chem., 1983, 1656; Reichenbach, H., et al., Liebigs Ann. Chem., 1984, 1088; Reichenbach, H., et al., Liebigs Ann. Chem., 1985, 822. The structures of these compounds are closely related having in common a 3-acyl-4-hydroxy-2-pyrone with an alkyl chain at the 6-position bearing a vinyl carbamate functionality, a feature atypical of natural products. They differ only in the substitution on the alkyl chain attached to the 3-position of the pyrone, the corallopyronins being more elaborate. These compounds are further disclosed in U.S. Pat. No. 6,228,882, PCT/US99/00303, PCT/US97/05991, and PCT/US99/21544.

Other RNAP inhibitors suitable for purposes of the present invention are disclosed in the patent literature, including:
dioxolane and oxathiolane derivatives, PCT/US02/28078;
Nucleoside derivatives, PCT/US02/01531, PCT/US02/03086,
synthetic L-peptides, PCT/US99/04351;
phage T7 lysozyme, PCT/US94/05409; and
arylhydrazones, U.S. Pat. No. 5,760,063.

The term “treating” as used herein refers to the alleviation of symptoms of a particular disorder in a patient or the improvement of an ascertainable measurement associated with a particular disorder. As used herein, the term “patient” refers to a mammal, including a human.

The terms “HBV”, “HCV” and “HGV” refer to hepatitis-B, hepatitis-C and hepatitis-G virus, respectively.

In practicing the present invention, many techniques in molecular biology, microbiology, recombinant DNA, and protein biochemistry such as are referred to herein are explained fully in, for example, Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984, (M. L. Gait ed.); Transcription and Translation, 1984 (Hames and Higgins eds.); A Practical Guide to Molecular Cloning; the series, Methods in Enzymology (Academic Press, Inc.); and Protein Purification: Principles and Practice, Second Edition (Springer-Verlag, N.Y.).

Routes of Administration and Dosage Forms

The dosage administered will, of course, vary depending upon known factors, such as the pharmacodynamic characteristics of the particular agent and its mode and route of administration; the age, health and weight of the recipient; the nature and extent of the symptoms, the kind of concurrent treatment; the frequency of treatment; and the effect desired. A daily dosage of active ingredient can be expected to be about 0.001 to 1000 milligram (mg) per kilogram (kg) of body weight, with the preferred dose being 0.1 to about 30 mg/kg.

Dosage forms (compositions suitable for administration) contain from about 1 mg to about 100 mg of active ingredient per unit. In phase pharmaceutical compositions, the active ingredient will ordinarily be present in an amount of about 0.5-95% by weight based on the total weight of the composition.

The present invention provides compositions comprising RNAP and IMPDH inhibitors, as well as methods of combating viral infection comprising administering said compositions and/or administering the RNAP and IMPDH inhibitors separately, either simultaneously or consecutively. In a preferred embodiment, the IMPDH inhibitor is a mycophenolate or ribavirin.

The compounds and compositions according to the invention may be administered by any suitable therapeutic route including oral, rectal, nasal, topical (including transdermal, buccal and sublingual), vaginal and parenteral (including subcutaneous, intramuscular, intravenous and intradermal). It will be appreciated that the preferred route will vary with the condition and age of the recipient, the nature of the infection and the chosen active ingredient.

In an embodiment of the invention, the RNAP and IMPDH inhibitors are administered substantially simultaneously. In an embodiment, a second inhibitor (either of an RNAP or a IMPDH inhibitor) is administered after a medically appropriate period following administration of a first inhibitor (either of an RNAP inhibitor or an IMPDH inhibitor). In an embodiment, the RNAP and IMPDH inhibitors are administered by the same route of administration. In a further embodiment, the RNAP and IMPDH inhibitors are administered by separate routes of administration. Skilled practitioners of the medical arts are able to determine appropriate routes and schedules of administration.

Pharmaceutical formulations of the present invention include those suitable for oral, rectal, nasal, topical (including transdermal buccal and sublingual), vaginal or parenteral (including subcutaneous, intramuscular, intravenous and intradermal) administration.

The formulations may conveniently be presented in unit dosage form and may be prepared by methods known in the art of pharmacy. Such methods include the step of bringing into association the RNAP and IMPDH inhibitors with the carrier. The optional carrier comprises one or more accessory ingredients. In general, the formulations are prepared by uniformly and intimately bringing into association the RNAP and IMPDH inhibitors with liquid carriers or finely divided solid carriers or both, and then if necessary shaping the product.

Compositions suitable for transdermal administration may be presented as discrete patches adapted to remain in intimate contact with the epidermis of the recipient for a prolonged period of time. Such patches suitably contain the RNAP and IMPDH inhibitors in an optionally buffered, aqueous solution, or dissolved and/or dispersed in an adhesive, or dispersed in a polymer. A suitable concentration of each RNAP and IMPDH inhibitor is about 1% to 25%, preferably about 5 to 15%. The active compounds may also be delivered from the patch by electrotransport or iontophoresis as generally described in Pharmaceutical Research, 3 (6), 318 (1986) (the entire disclosure of which is incorporated herein by reference).

Formulations of the present invention suitable for oral administration may be presented as discrete units such as capsules, caches or tablets, each containing a predetermined amount of the RNAP and IMPDH inhibitors as a powder or granules; as a solution or a suspension in an aqueous or non-aqueous liquid; or as an oil-in-water liquid emulsion or a water-in-oil liquid emulsion. The RNAP and IMPDH inhibitors may also be presented as a bolus, electuary or paste.

A tablet may be made by compression or molding, optionally with one or more accessory ingredients. Compressed tablets may be prepared by compressing, in a suitable machine, the RNAP and IMPDH inhibitors in a free-flowing form such as a powder or granules, optionally mixed with a binder (e.g. povidone, gelatin, carboxymethyl cellulose, or hydroxypropylmethyl cellulose, as non-limiting examples known to the art), lubricant, inert diluent, preservatives, disintegrant (e.g. sodium starch glycollate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of one or more of the inhibitor ingredients therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising one or more of the synergistic ingredients in a flavored basis, usually sucrose and acacia or tragacanth; pastilles comprising one or more of the inhibitor ingredients in an inert basis such as gelatin and glycerin, or sucrose and acacia; and mouthwashes comprising the one or more of the inhibitor ingredients in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing, in addition to the one or more RNAP and IMPDH inhibitors, such carriers as are known in the art to be appropriate.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multidose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as described herein, or an appropriate fraction thereof, of one or more of the RNAP and IMPDH inhibitors.

It should be understood that, in addition to the ingredients particularly mentioned above, the formulations of this invention may include other agents conventional in the art having regard to the type of formulation in question; for example, those suitable for oral administration may include such further agents as sweeteners, thickeners and flavoring agents.

The foregoing disclosure includes all the information deemed essential to enable those skilled in the art to practice the claimed invention. Because the cited applications may provide further useful information, these cited materials are hereby incorporated by reference in their entirety.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

### INCORPORATION BY REFERENCE

All publications and patent applications cited in this specification are herein incorporated by reference, and for any and all purposes, as if each individual publication or patent application were specifically and individually indicated to be incorporated by reference. In the case of inconsistencies the present disclosure will prevail.

1. A pharmaceutical composition comprising:
at least one RNA polymerase inhibitor; and
at least one inosine monophosphate dehydrogenase (IMPDH) inhibitor, wherein the combination provides an effective treatment of a viral infection.

2. A pharmaceutical composition, according to claim 1, wherein said IMPDH inhibitor is selected from the group consisting of mycophenolates, ribavirin, tiazofurin, mizoribineVX-497, brenidin, a substituted urea, a substituted thiourea, a substituted sulfonylurea, a phenyl thiourea, a heterocyclic carboxamide thiourea, an oxamide, a tetraphosphonate bicyclic trisanhydride, a substituted oxazole, a nucleoside analogue, an aminoquinoline, rapamycin, a polycyclic secondary amine, and C-nucleoside isosteres of nicotinamide adenine dinucleotide.

3. A pharmaceutical composition, according to claim 1, wherein said IMPDH inhibitor is a mycophenolate.

4. A pharmaceutical composition, according to claim 1, wherein said IMPDH inhibitor is ribavirin.

5. A pharmaceutical composition, according to claim 1, wherein said RNAP inhibitor is selected from the group consisting of amanitin, rifampicin, actinomycin D, streptolydigin, doxorubicin, Tagetin®, a corallopyronin, and a myxopyronin.

6. A pharmaceutical composition, according to claim 1, wherein said RNAP inhibitor is Tagetin®.

7. A method of treating a viral infection comprising: administering to infected cells a pharmaceutical composition comprising:
a) an amount of at least one RNA polymerase inhibitor; and
b) an amount of at least one IMPDH inhibitor, wherein the combination provides an effective treatment of a viral infection.

8. A method of treating a viral infection comprising:
administering to an animal in need of such treatment a pharmaceutical composition comprising an amount of at least one RNA polymerase inhibitor; and subsequently
administering to an animal in need of such treatment a pharmaceutical composition comprising an amount of at least one IMPDH inhibitor, wherein the combination provides an effective treatment of a viral infection.

9. A method of treating a viral infection comprising:
administering to an animal in need of such treatment a pharmaceutical composition comprising an amount of at least one IMPDH inhibitor; and subsequently
administering to an animal in need of such treatment a pharmaceutical composition comprising an amount of at least one RNA polymerase inhibitor, wherein the combination provides an effective treatment of a viral infection.

10. A method of manufacturing a pharmaceutical composition comprising bringing into association with a pharmaceutical carrier:
an amount of at least one RNA polymerase inhibitor; and
an amount of at least one IMPDH inhibitor, wherein the combination provides an effective treatment of a viral infection.
